(19) United States
(12) Patent Application Publication
(10) Pub. No.: US 2020/0265578 A1
(43) Pub. Date: Aug. 20, 2020

(54) SYSTEM AND METHOD FOR UTILIZING GENERAL-PURPOSE GRAPHICS PROCESSING UNITS (GPGPU) ARCHITECTURE FOR MEDICAL IMAGE PROCESSING

(71) Applicant: THE GENERAL HOSPITAL CORPORATION, Boston, MA (US)

(72) Inventor: Synho DO, Lexington, MA (US)

(21) Appl. No.: 16/645,024

(22) PCT Filed: Sep. 7, 2018
(86) PCT No.: PCT/US2018/049953

§ 371 Date: Mar. 6, 2020

Related U.S. Application Data
(60) Provisional application No. 62/555,730, filed on Sep. 8, 2017.

(51) Int. Cl. G06T 7/00; G06T 1/20; G06N 3/04; A61B 6/03; A61B 6/00

(52) U.S. Cl. CPC G06T 7/0012; G06T 1/20; G06N 3/04; A61B 6/037; A61B 6/032; G06T 2207/20084; G06T 2207/10081; G06T 2207/10088; G06T 2207/10104; G06T 2207/10132; A61B 6/5211

(57) ABSTRACT

Systems and methods for translating medical imaging data for processing using a general processing graphic processing unit (GPGPU) architecture are provided. Medical imaging data acquired from a patient and having data characteristics incompatible with processing on the GPGPU architecture, including at least one of bit-resolution, memory capacity requirements for processing, or bandwidth requirements for processing is translated for processing by the GPGPU architecture. The translation process is performed by determining a plurality of window level settings using a machine learning network to increase conspicuity of an object in an image generated from the medical imaging data or generate at least two channel image datasets from the medical imaging data. Translated medical image data is crated using at least one of the window level settings or at least two channel image datasets and then processed using the GPGPU architecture to generate medical images of the patient.

### CROSS-REFERENCE TO RELATED APPLICATIONS

This application claims the benefit of U.S. Provisional Patent Application Ser. No. 62/555,730 filed on Sep. 8, 2017 and entitled “Multi-bit Resolution and Multi-scale Medical Image Machine Learning Solution with GPGPU Architecture.”

### STATEMENT REGARDING FEDERALLY SPONSORED RESEARCH

### BACKGROUND

General-purpose graphics processing units (GPGPU) use graphics processing units (GPU) to perform manipulations or computations on images. Traditionally, image computations were performed using conventional central processing units (CPU), but the parallel computing power of GPUs and their ability to efficiently analyze image data has provided recent motivation for using GPUs in the medical imaging industry.

GPGPUs, however, are often optimized for single precision computation with massive parallel computation units, not for double precision bit-resolution, which may be more common in medical imaging (for example, 16-bit DICOM format, floating point computation, etc.). GPGPUs also have limited on-board memory capacity and bandwidth, making processing large dataset not feasible. For example, using GPGUs to process a large-matrix, such as chest X-ray that could include >2000×3000 pixels; 3D volume data set provided by computed tomography (CT), positron emission tomography (PET), or magnetic resonance imaging (MRI); or time-resolved 2D images from ultrasound, perfusion CT, or CINE studies not feasible.

Therefore, it would be desirable to have systems and methods for processing the large and extensive datasets generated by medical imaging studies using efficiency and flexibility provided by specialized processing units.

### SUMMARY OF THE DISCLOSURE

The present disclosure addresses the aforementioned drawbacks by providing a system and method for multi-bit resolution and multi-scale medical image processing that allows for general processing of the large datasets of medical images with highly-specialized processing systems of GPUs (i.e., the systems and methods provided herein provide a GPGPU architecture). A machine-learning architecture is provided that facilitates the multi-bit resolution and multi-scale medical image processing using specialized processing systems, such as GPUs in a general processing function (i.e., GPGPU). The provided systems and methods impart the ability to process images with subtle changes, such as images of soft-tissue organs (e.g., liver, kidney, brain, and the like) and functional images with contrast materials (e.g., iodine, gadolinium, and the like) with efficiency and effectiveness not realized with traditional CPU processing or non-general processing using a GPU. In some configurations, image window settings may be dynamically optimized using machine learning to create reformatted images that increase conspicuity of image pathologies.

In accordance with one aspect of the present disclosure, a method is provided for configuring medical imaging data acquired from a patient for processing using a general processing graphic processing unit (GPGPU) architecture. The method includes acquiring medical imaging data acquired from a patient using at least one of a magnetic resonance imaging (MRI) system, a computed tomography (CT) system, an ultrasound system, or a positron emission tomography (PET) system and having data characteristics incompatible with processing on the GPGPU architecture, including at least one of bit-resolution, memory capacity requirements for processing, or bandwidth requirements for processing. The method also includes subjecting the medical imaging data to a system for translating medical imaging data for processing by the GPGPU architecture. The system for translating the medical imaging data is configured to determine a plurality of window level settings using a machine learning network to increase conspicuity of an object in an image generated from the medical imaging data or generate at least two channel image datasets from the medical imaging data and create translated medical image data using at least one of the window level settings or at least two channel image datasets. The method also includes processing the translated medical image data using the GPGPU architecture to generate medical images of the patient and displaying the medical images of the patient.

In accordance with another aspect of the present disclosure, a system for translating medical imaging data acquired from a patient for processing using a general processing graphic processing unit (GPGPU) architecture. The system includes a first processor configured to acquire medical imaging data acquired from a patient and having data characteristics incompatible with processing on the GPGPU architecture, including at least one of bit-resolution, memory capacity requirements for processing, or bandwidth requirements for processing. The first processor is further configured to translate medical imaging data for processing by the GPGPU architecture by determining a plurality of window level settings using a machine learning network to increase conspicuity of an object in an image generated from the medical imaging data or generate at least two channel image datasets from the medical imaging data and creating translated medical image data using at least one of the window level settings or at least two channel image datasets. The system also includes a second processor having a GPU architecture configured to process the translated medical image data using the GPGPU architecture to generate medical images of the patient and a display configured to display the medical images of the patient generated by the GPGPU architecture

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a schematic diagram of one system in accordance with the present disclosure.

FIG. 2 is a schematic diagram showing further details of one, non-limiting example of the system of FIG. 1.

FIG. 3 is a flowchart setting forth some non-limiting examples of steps for one configuration of reformatting images for processing using a GPGPU architecture in accordance with the present disclosure.

FIG. 4 is a flowchart setting forth some non-limiting examples of steps for one configuration of reformatting images using machine learning to dynamically optimize window level settings in accordance with the present disclosure.

FIG. 5 is a schematic for one configuration of window leveling an original image into multiple color channels that are then combined for creating a reformatted image in accordance with the present disclosure.

FIG. 6 is a schematic for one configuration of a dynamic window optimization process in accordance with the present disclosure.

### DETAILED DESCRIPTION

Systems and methods are provided for multi-bit resolution and multi-scale medical image machine learning processing that allows medical image processing to be compatible with a general-purpose graphics processing unit (GPU) (GPGPU) architecture. In one configuration, the machine learning processing may be used to reformat high definition medical images to facilitate processing of the medical images on a GPGPU architecture. In one configuration, the machine learning processing may be used for dynamic window setting optimization to increase conspicuity of pathology found in the images.

A GPGPU is a GPU that performs non-specialized calculations that would typically be conducted by the central processing unit (CPU). Ordinarily, the GPU is dedicated to graphics rendering and, as a result, GPUs are highly-specialized for graphics rendering and aren't amenable to general processing that has been the domain of the CPU. However, because GPUs are constructed for massive parallelism, they can dwarf the calculation rate of even the most powerful CPUs so long as the task being performed is designed for or amenable to parallel processing. Unfortunately, medical imaging does not, traditionally, fit into the GPU processing paradigm.

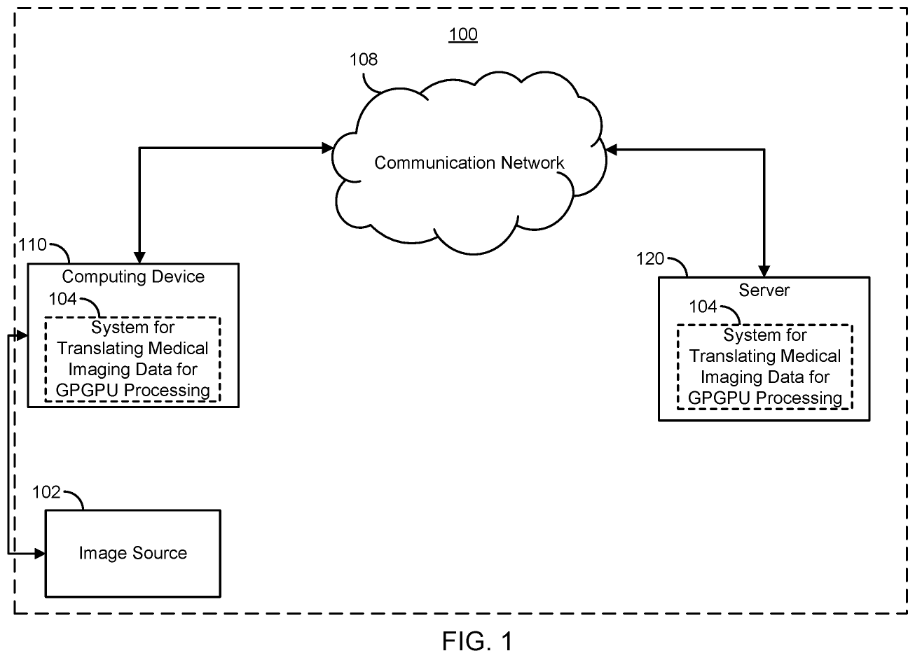

Referring to FIG. 1, an example of a system 100 in accordance with some aspects of the disclosed subject matter is provided. As shown in FIG. 1, a computing device 110 can receive multiple types of image data from an image source 102. In some configurations, the computing device 110 can execute at least a portion of a system for translating data for GPGPU processing 104. That is, as described above, medical imaging data, such as acquired from an MRI, CT, ultrasound, PET, or other modality is generally not compatible with processing using a GPGPU. Thus, the system 100 provides a system for translating data for GPGPU processing 104, as will be described.

Additionally or alternatively, in some configurations, the computing device 110 can communicate information about image data received from the image source 102 to a server 120 over a communication network 108, which can also include a version of a system for translating data for GPGPU processing 104.

In some configurations, the computing device 110 and/or server 120 can be any suitable computing device or combination of devices, such as a desktop computer, a laptop computer, a smartphone, a tablet computer, a wearable computer, a server computer, a virtual machine being executed by a physical computing device, etc.

In some configurations, the image source 102 can be any suitable source of medical image data, such as an MRI, CT, ultrasound, PET, SPECT, x-ray, or another computing device (e.g., a server storing image data), and the like. In some configurations, the image source 102 can be local to the computing device 110. For example, the image source 102 can be incorporated with the computing device 110 (e.g., the computing device 110 can be configured as part of a device for capturing and/or storing images). As another example, the image source 102 can be connected to the computing device 110 by a cable, a direct wireless link, or the like. Additionally or alternatively, in some configurations, the image source 102 can be located locally and/or remotely from the computing device 110, and can communicate image data to the computing device 110 (and/or server 120) via a communication network (e.g., the communication network 108).

In some configurations, the communication network 108 can be any suitable communication network or combination of communication networks. For example, the communication network 108 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some configurations, the communication network 108 can be a local area network, a wide area network, a public network (e.g., the Internet), a private or semi-private network (e.g., a corporate or university intranet), other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

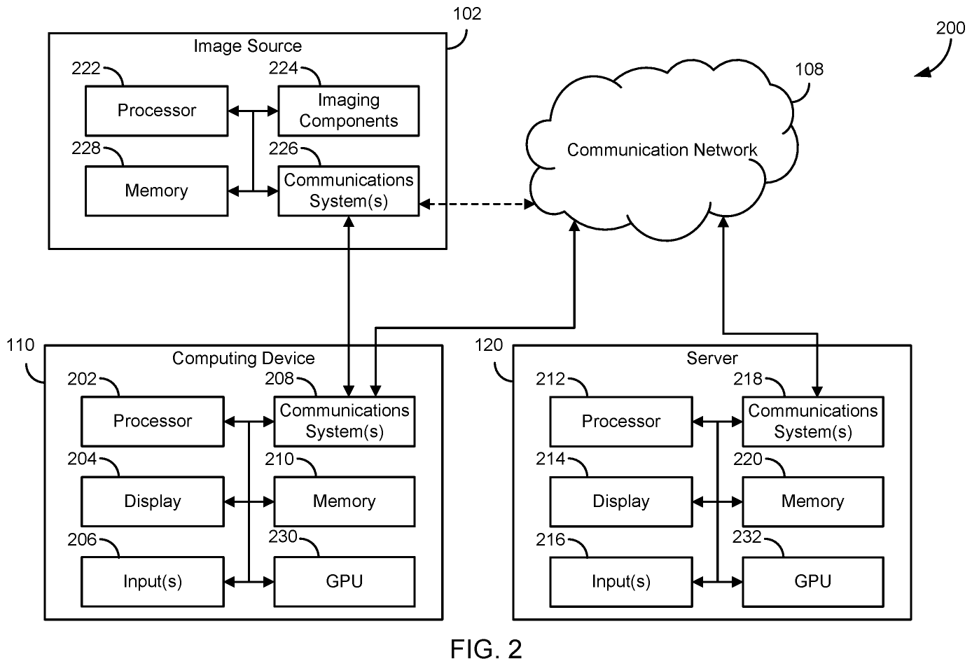

FIG. 2 shows an example of hardware 200 that can be used to implement the image source 102, computing device 110, and/or server 120 in accordance with some aspects of the disclosed subject matter. As shown in FIG. 2, in some configurations, the computing device 110 can include a processor 202, a display 204, one or more inputs 206, one or more communication systems 208, memory 210, and/or a GPU 230. In some configurations, the processor 202 can be any suitable hardware processor or combination of processors, such as a central processing unit (CPU). In some configurations, the display 204 can include any suitable display devices, such as a computer monitor, a touchscreen, a television, etc. In some configurations, the inputs 206 can include any of a variety of suitable input devices and/or sensors that can be used to receive user input, such as a keyboard, a mouse, a touchscreen, a microphone, and the like.

In some configurations, the communications systems 208 can include a variety of suitable hardware, firmware, and/or software for communicating information over the communication network 108 and/or any other suitable communication networks. For example, the communications systems 208 can include one or more transceivers, one or more communication chips and/or chip sets, etc. In a more particular example, the communications systems 208 can include hardware, firmware and/or software that can be used to establish a Wi-Fi connection, a Bluetooth connection, a cellular connection, an Ethernet connection, etc.

In some configurations, the memory 210 can include any suitable storage device or devices that can be used to store instructions, values, etc., that can be used, for example, by the processor 202 to present content using the display 204, to communicate with the server 120 via the communications system(s) 208, and the like. The memory 210 can include any of a variety of suitable volatile memory, non-volatile memory, storage, or any suitable combination thereof. For example, the memory 210 can include RAM, ROM, EEPROM, one or more flash drives, one or more hard disks, one or more solid state drives, one or more optical drives, etc. In some configurations, the memory 210 can have encoded thereon a computer program for controlling operation of the computing device 110. In such configurations, the processor 202 can execute at least a portion of the computer program to present content (e.g., MRI images, user interfaces, graphics, tables, and the like), receive content from the server 120, transmit information to the server 120, and the like.

In some configurations, the server 120 can include a processor 212, a display 214, one or more inputs 216, one or more communications systems 218, memory 220, and/or GPU 232. In some configurations, the processor 212 can be a suitable hardware processor or combination of processors, such as a CPU, and the like. In some configurations, the display 214 can include a suitable display devices, such as a computer monitor, a touchscreen, a television, and the like. In some configurations, the inputs 216 can include a suitable input devices and/or sensors that can be used to receive user input, such as a keyboard, a mouse, a touchscreen, a microphone, and the like.

In some configurations, the communications systems 218 can include a suitable hardware, firmware, and/or software for communicating information over the communication network 108 and/or any other suitable communication networks. For example, the communications systems 218 can include one or more transceivers, one or more communication chips and/or chip sets, and the like. In a more particular example, the communications systems 218 can include hardware, firmware and/or software that can be used to establish a Wi-Fi connection, a Bluetooth connection, a cellular connection, an Ethernet connection, and the like.

In some configurations, the memory 220 can include any suitable storage device or devices that can be used to store instructions, values, and the like, that can be used, for example, by the processor 212 to present content using the display 214, to communicate with one or more computing devices 110, and the like. The memory 220 can include any of a variety of suitable volatile memory, non-volatile memory, storage, or any suitable combination thereof. For example, the memory 220 can include RAM, ROM, EEPROM, one or more flash drives, one or more hard disks, one or more solid state drives, one or more optical drives, and the like. In some configurations, the memory 220 can have encoded thereon a server program for controlling operation of the server 120. In such configurations, the processor 212 can execute at least a portion of the server program to transmit information and/or content (e.g., MRI data, results of automatic diagnosis, a user interface, and the like) to one or more computing devices 110, receive information and/or content from one or more computing devices 110, receive instructions from one or more devices (e.g., a personal computer, a laptop computer, a tablet computer, a smartphone, and the like), and the like.

In some configurations, the image source 102 can include a processor 222, imaging components 224, one or more communications systems 226, and/or memory 228. In some embodiments, processor 222 can be any suitable hardware processor or combination of processors, such as a CPU and the like. In some configurations, the imaging components 224 can be any suitable components to generate image data corresponding to one or more imaging modes (e.g., T1 imaging, T2 imaging, fMRI, and the like). An example of an imaging machine that can be used to implement the image source 102 can include a conventional MRI scanner (e.g., a 1.5 T scanner, a 3 T scanner), a high field MRI scanner (e.g., a 7 T scanner), an open bore MRI scanner, a CT system, an ultrasound scanner, a PET system, and the like.

Note that, although not shown, the image source 102 can include any suitable inputs and/or outputs. For example, the image source 102 can include input devices and/or sensors that can be used to receive user input, such as a keyboard, a mouse, a touchscreen, a microphone, a trackpad, a trackball, hardware buttons, software buttons, and the like. As another example, the image source 102 can include any suitable display devices, such as a computer monitor, a touchscreen, a television, etc., one or more speakers, and the like.

In some configurations, the communications systems 226 can include any suitable hardware, firmware, and/or software for communicating information to the computing device 110 (and, in some embodiments, over the communication network 108 and/or any other suitable communication networks). For example, the communications systems 226 can include one or more transceivers, one or more communication chips and/or chip sets, and the like. In a more particular example, the communications systems 226 can include hardware, firmware and/or software that can be used to establish a wired connection using any suitable port and/or communication standard (e.g., VGA, DVI video, USB, RS-232, and the like), Wi-Fi connection, a Bluetooth connection, a cellular connection, an Ethernet connection, and the like.

In some configurations, the memory 228 can include any suitable storage device or devices that can be used to store instructions, values, image data, and the like, that can be used, for example, by the processor 222 to: control the imaging components 224, and/or receive image data from the imaging components 224; generate images; present content (e.g., MRI images, a user interface, and the like) using a display; communicate with one or more computing devices 110; and the like. The memory 228 can include any suitable volatile memory, non-volatile memory, storage, or any of a variety of other suitable combination thereof. For example, the memory 228 can include RAM, ROM, EEPROM, one or more flash drives, one or more hard disks, one or more solid state drives, one or more optical drives, and the like. In some configurations, the memory 228 can have encoded thereon a program for controlling operation of the image source 102. In such configurations, the processor 222 can execute at least a portion of the program to generate images, transmit information and/or content (e.g., MRI image data) to one or more the computing devices 110, receive information and/or content from one or more computing devices 110, receive instructions from one or more devices (e.g., a personal computer, a laptop computer, a tablet computer, a smartphone, and the like), and the like.

In some configurations, image source 102 may generate any format of medical image data, such as an MRI, CT, ultrasound, PET, SPECT, x-ray, and the like. Medical image data includes not only data for reconstructing the image itself, which may be compressed or not, but also contains patient identification and demographic information and technical information about the exam, including image series data, acquisition or protocol information, and other details. Medical image data may also be in the form of complex image series information, such as time-resolved 2D image series, 3D volumes, and may include additional information, such as elastography data on tissue stiffness or other diagnostic notations. The complexity and size of medical image data prevents traditional CPUs from being able to efficiently and effectively manipulate medical images or processing medical images for movement on a network.

As previously described, the GPUs 230 and 232 are optimized for graphics processing. For example, the GPUs 230 and/or 232 may be designed for single precision computation with massive parallel computation units, not for double precision bit-resolution, which is common in medical imaging (for example, 16-bit DICOM format, floating point computation, etc.). The GPUs 230 and/or 232 may also have limited on-board memory capacity and bandwidth, making processing large dataset not feasible. For example, using GPGUs to process a large-matrix, such as chest X-ray that could include >2000×3000 pixels; 3D volume data set provided by computed tomography (CT), positron emission tomography (PET), or magnetic resonance imaging (MRI); or time-resolved 2D images from ultrasound, perfusion CT, or CINE studies not feasible.

Thus, the present disclosure provides a system for translating medical imaging data to be compatible with GPGPU processing. In particular, as will be described, image data acquired with image source 102 may be processed using a specially-designed machine learning system to enable the medical imaging data to be processed using the GPGPU 230 and/or 232. For example, the present disclosure provides systems and methods that enable initial image processing and/or image reconstruction to be performed by the computing device 110 using the GPU 230, or by the server 120 using the GPU 232.

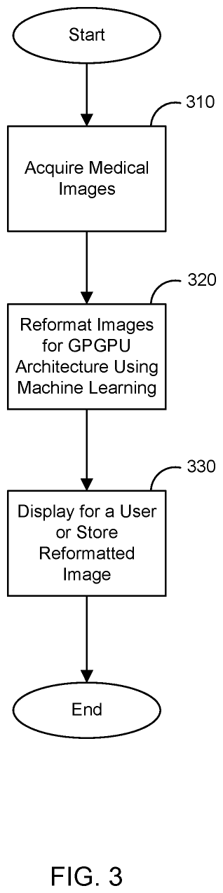

Referring to FIG. 3, the system for translating medical imaging data for GPGPU processing 104 may be designed to reformat medical imaging data for GPGPU processing. Such a process starts with acquiring medical imaging data at step 310. Medical imaging data may be acquired by the image source 102 described above with FIG. 1, and/or may be acquired from an image storage system, such as a PACS system. Reformatting the images for GPGPU architecture is performed at step 320 using a machine learning (ML) routine. The ML algorithm may use seek to maintain the high bit-resolution resolution of the underlying medical imaging data without losing fast computation capability provided by GPGPU processing. Reformatting may be done on high definition medical imaging data to enable processing on a GPGPU architecture. High definition formats may be dynamically converted to maximize the utilization of GPU architecture. Reformatted and processed images may then be displayed for a user or stored in an image storage system, such as a PACS, at step 330.

Reformatting may include using machine learning to separate or break apart an original image into different resolutions or channels, as described below. A multi-resolution approach may allow for processing large image datasets or images while maintaining hardware efficiency. In some configurations, the system for translating medical imaging data for GPGPU processing may be deployed on a dedicated, fast FPGA and/or GPU bit-resolution conversion system.

Dynamically conversion may include performing the reformatting in an adaptable way, such that the number and form of the channels selected may be adjusted based upon user feedback, previous training of the machine learning routine, defined requirements for the final reconstructed image (such as a window level, contrast, signal to noise ratios, image kernels, and the like), original image information (such as imaging modality used, clinical task to be performed, window level, contrast, signal to noise ratios, image kernels, and the like), similar characteristics or priorities, or a combination thereof

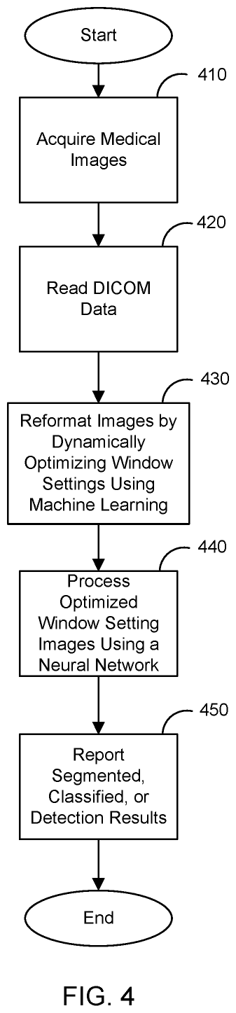

Referring to FIG. 4, a flowchart for one configuration of reformatting images for GPGPU architecture starts with acquiring medical images at step 410. DICOM header data for the images may be read at step 420. At step 430, window level settings may be dynamically optimized using a machine learning architecture. The reformatted and window level optimized image data may then be further processed by an artificial intelligence network, such as a convolutional neural network, at step 440. The neural network may process the images by segmenting the images, detecting abnormalities, or regions of interest in the images, classifying regions or objects in the images, and the like. The results of this neural network processing may be reported, such as to deliver the imaging data to for GPGPU processing and/or such as to communicate the results of the reformatting, segmenting, classification, or detection results a user at step 450.

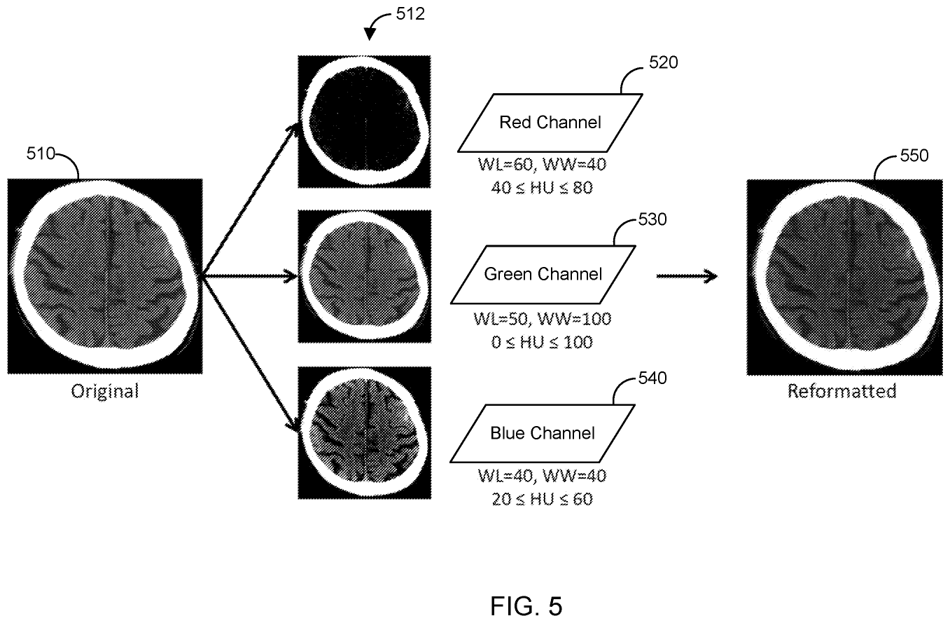

Referring to FIG. 5, image reformatting may include applying different window/level settings to the original input image 510 in order to generate multiple different channel images 512. Each channel image can be generated by applying a specified window/level setting to pixels in the input image 510. For instance, a specified window/level setting can be applied to pixels having intensity values in a specified range associated with the specified window. As another example, a specified window/level setting can be applied to pixels having quantitative values, such as Hounsfield Units (HU) or other intensity values, within a specified range associated with the specified window. Any number of channel images may be generated, and a reformatted image 550 can be created by combining the channel images.

In some implementations, the different channel images may be colorized, such as mapping pixel intensity values in the channel images to one or more different color scales, or by otherwise assigning a specific color to each different channel image. For example, a red channel image 520 may be generated using a window/level setting with a window level (WL)=60 and window width (WW)=40 for pixels in the input image 510 corresponding to HU values in the range of 40-80. The pixel values in the red channel image 520 are then assigned a suitable RGB value, such as by mapping the pixel values to an RGB color scale. A green channel image 530 may be generated using a window/level setting with a level WL=50 and a window WW=100 for pixels in the input image 510 corresponding to HU values in the range of 0-100. The pixel values in the green channel image 530 are then assigned a suitable RGB value, such as by mapping the pixel values to an RGB color scale. A blue channel image 540 may be generated using a window/level setting with a level WL=40 and a window WW=40 for pixels in the input image 510 corresponding to HU values in the range of 20-60. The pixel values in the blue channel image 540 are then assigned a suitable RGB value, such as by mapping the pixel values to an RGB color scale. When the different channel images are assigned different colors (e.g., by converting grayscale values to RGB values, or values from a different colormap or color scale), the reformatted image 550 may be stored or presented to a user as a multi-color image. In some instances, the channel images can be individually processed via GPGPU processing and then combined to form a combined image (e.g., an RGB image when combining red, green, and blue channel images).

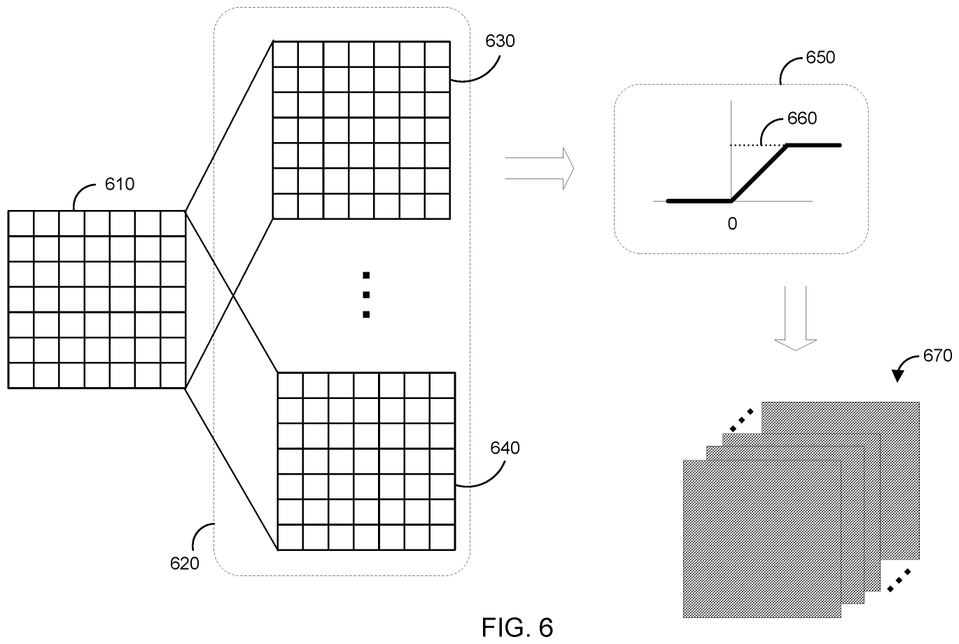

Referring to FIG. 6, a schematic is shown for one configuration of a dynamic window optimization process. A window block 610 may be processed using a convolutional kernel 620, such as a 1×1 convolutional kernel as shown in FIG. 6. The convolutional kernel 620 may include n channels, where at least one element of first block 630 may be selected for one channel and at least one element of nth block 640 may be selected for an nth channel. Any number of channels may be selected. First block 630 may be considered a neuron, Y, in a neural network, which may be determined by Y=wₙx+bₙ where w is a weight, n is the channel number and b is a bias. Y is activated (or a channel is determined) when exceeding an activation threshold. An activation threshold or function may be selected by a user prior to processing, such as a linear function, a tanh function, a sigmoid, a ReLU, a leaky ReLU, or any other desirable or appropriate activation function. In one example, an ReLU function may be selected as the activation function. The ReLU function provides an output x if x is positive and 0 otherwise, as indicated by: A(x)=max(0, x). By providing an upper bound 660, the bound ReLU function 650 may prevent the activation function from blowing up, which would result in a nonfunctional analysis. Channels may be selected as described above using the results from the activation function. Windowed images 670 may then be ready for further processing, as described above.

In addition to processing medical imaging data for GPGPU processing, the systems and methods described above may be designed to also process the medical imaging data to optimize processing for a particular clinical application, such as to improve or optimize contrast for a particular clinical study. Such considerations for clinical application can be achieved simultaneously and/or in parallel with the above-described process for preparing medical imaging data for processing using a GPGPU architecture. For example, window processing or channel processing may be performed to facilitate processing of the medical imaging data using a GPGPU and also to improve contrast or format the images for a particular clinical imaging study. As a non-limiting examples, clinical applications of intracranial hemorrhaging studies, muscle segmentation, stone classification, and mammography density, will be described. However, any of a wide variety of clinical applications are likewise applicable by applying the same or similar implementations of the described systems and methods.

### Example for Intracranial Hemorrhages

The systems and methods for image reformatting may be applied to preprocess images for a neural network configured to detect hemorrhages, such as intracranial hemorrhages. In some configurations, reformatting may increase the conspicuity for intracranial hemorrhages. Hemorrhages may include intracranial hemorrhage (ICH), an intraventricular hemorrhage (IVH), a subarachnoid hemorrhage (SAH), an intra-parenchymal hemorrhage (IPH), an epidural hematoma (EDH), a subdural hematoma (SDH), or a bleed. An overview of an example dataset where some images where used for training, validation, and testing of a neural network's ability to detect and classify various forms of hemorrhages is shown in Table 1 and Table 2, where windowed image refers to the reformatted image and full-range DICOM image refers to an original image with 1 input channel used for all cases. AUC is area under the curve.

In some configurations, optimization of the number of channels may be performed by determining the AUC corresponding to the upbound value for a test channel number. In one example, for an upbound value of 255, the area under the curve varies with the number of channels used (number of channels:AUC): 1:0.950; 2:0.938; 3:0.962; 4:0.943; 5:0.939; 6:0.935; 7:0.925; 8:0.926; 9:0.937; 16:0.924; 32:0.939. Varying the upper bound for 3 channels in the present example reflects AUCs of (upbound value:AUC): 1:0/831; 6:0.900; 255:0.962; 511:0.936; 1023:0.934; 2047:0.897. This reflects a peak AUC value for the upbound value of 255.

TABLE 1

| | Train | | Validation | | Test | |
|---|---|---|---|---|---|---|
| | # Cases | # Slices | # Cases | # Slices | # Cases | # Slices |
| No ICH | 141 | 2202 | 30 | 474 | 30 | 475 |
| ICH | 337 | 1915 | 91 | 490 | 91 | 475 |
| IPH | 220 | 1032 | 44 | 240 | 44 | 238 |
| IVH | 75 | 306 | 17 | 89 | 17 | 85 |
| SAH | 153 | 577 | 30 | 161 | 30 | 152 |
| Total | 478 | 4117 | 121 | 964 | 121 | 950 |

TABLE 2

| | DWO Parameters | | | Test Performance | | |
|---|---|---|---|---|---|---|
| Input Image | Init | # Channels | Upbound | AP | AUC | # Model |
| Windowed | | | | 0.915 | 0.955 | 29 (lr = 0.001) |
| Image | | | | | | |
| Full-range | | | | 0.547 | 0.850 | 21 (lr = 0.01) |
| DICOM | No | 1 | 255 | 0.874 | 0.936 | 29 (lr = 0.001) |
| | No | 3 | 255 | 0.910 | 0.956 | 29 (lr = 0.01) |
| | No | 5 | 255 | 0.845 | 0.939 | 26 (lr = 0.01) |
| | Yes | 1 | 255 | 0.932 | 0.964 | 29 (lr = 0.001) |
| | Yes | 3 | 255 | 0.919 | 0.958 | 26 (lr = 0.01) |
| | Yes | 5 | 255 | 0.921 | 0.959 | 24 (lr = 0.01) |
| | Yes | 7 | 255 | 0.893 | 0.946 | 29 (lr = 0.01) |
| | Yes | 9 | 255 | 0.918 | 0.962 | 39 (lr = 0.01) |
| | Yes | 16 | 255 | 0.923 | 0.958 | 39 (lr = 0.01) |
| | Yes | 32 | 255 | 0.867 | 0.938 | 31 (lr = 0.01) |

### Example for Muscle Segmentation

The systems and methods for image reformatting may be applied to preprocess images for a neural network configured to segment muscles in medical images. In some configurations, reformatting may increase the conspicuity for muscles in the images. An overview of an example dataset where some images where used for training, validation, and testing of a neural network's ability to segment muscles is shown in Table 3 and Table 4, where windowed image refers to the reformatted image and full-range DICOM image refers to an original image with 1 input channel used for all cases.

TABLE 3

| Train | | Validation | | Test | |
|---|---|---|---|---|---|
| # Cases | # Slices | # Cases | # Slices | # Cases | # Slices |
| 240 | 250 | 50 | 50 | 150 | 150 |

TABLE 4

| | DWO Parameters | | | Test Performance | | |
|---|---|---|---|---|---|---|
| Input Image | Init | # Chanls | Upbound | Dice | IoU | # Model |
| Windowed | | | | 0.93 ± 0.02 | N/A | |
| abdomen | | | | | | |
| Windowed | | | | 0.938 ± 0.031 | 0.885 ± 0.053 | 93 (lr = 1.0) |
| Abdomen | | | | | | |
| Full-range | | | | 0.906 ± 0.051 | 0.832 ± 0.078 | 96 (lr = 1.0) |
| DICOM | No | 1 | 255 | 0.941 ± 0.035 | 0.890 ± 0.053 | 81 (lr = 1.0) |
| | No | 3 | 255 | 0.951 ± 0.020 | 0.907 ± 0.036 | 81 (lr = 1.0) |
| | No | 5 | 255 | 0.950 ± 0.019 | 0.906 ± 0.034 | 99 (lr = 1.0) |
| | Yes | 1 | 255 | 0.947 ± 0.024 | 0.901 ± 0.042 | 85 (lr = 1.0) |
| | Yes | 3 | 255 | 0.951 ± 0.020 | 0.907 ± 0.035 | 64 (lr = 1.0) |
| | Yes | 5 | 255 | 0.950 ± 0.020 | 0.906 ± 0.036 | 99 (lr = 1.0) |

### Example for Stone Classification

The systems and methods for image reformatting may be applied to preprocess images for a neural network configured to classify stones in medical images. In some configurations, reformatting may increase the conspicuity for stones in the images. An overview of an example dataset where some images where used for training, validation, and testing of a neural network's ability to classify stones is shown in Table 5, Table 6, and Table 7, where windowed image refers to the reformatted image and full-range DICOM image refers to an original image with 1 input channel used for all cases. Table 6 depicts balanced test results, whereas Table 7 depicts all test results.

TABLE 5

| | Train | | Validation | | Test | |
|---|---|---|---|---|---|---|
| | # Cases | # Slices | # Cases | # Slices | # Cases | # Slices |
| No Stone | 176 | 1179 | 30 | 181 | 50 | 347 |
| GE | 118 | 408 | 15 | 86 | 25 | 139 |
| Siemens | 58 | 771 | 15 | 95 | 25 | 208 |
| Stone | 199 | 1179 | 30 | 181 | 50 | 347 |
| GE | 91 | 408 | 15 | 86 | 25 | 139 |
| Siemens | 108 | 771 | 15 | 95 | 25 | 208 |
| Total | 375 | 2358 | 121 | 362 | 121 | 694 |

TABLE 6

| | DWO Parameters | | | Test Performance | | |
|---|---|---|---|---|---|---|
| Input Image | Init | # Channels | Upbound | AP | AUC | # Model |
| Abdomen | | | | 0.871 | 0.867 | 37 (lr = 0.01) |
| Windowed | | | | | | |
| Bone | | | | 0.889 | 0.870 | 41 (lr = 0.01) |
| Windowed | | | | | | |
| Full-range | | | | 0.771 | 0.814 | 33 (lr = 0.001) |
| DICOM | No | 1 | 255 | 0.944 | 0.942 | 44 (lr = 0.01) |
| | No | 3 | 255 | 0.831 | 0.821 | 29 (lr = 0.01) |
| | No | 5 | 255 | 0.941 | 0.936 | 24 (lr = 0.01) |
| | Yes | 1 | 255 | 0.872 | 0.867 | 30 (lr = 0.01) |
| | Yes | 3 | 255 | 0.830 | 0.815 | 22 (lr = 0.005) |
| | Yes | 5 | 255 | 0.907 | 0.896 | 28 (lr = 0.005) |

TABLE 7

| | DWO Parameters | | | Test Performance | | |
|---|---|---|---|---|---|---|
| Input Image | Init | # Channels | Upbound | AP | AUC | # Model |
| Abdomen | | | | 0.737 | 0.942 | 23 (lr = 0.01) |
| Windowed | | | | | | |
| Bone | | | | 0.699 | 0.912 | 28 (lr = 0.01) |
| Windowed | | | | | | |
| Full-range | | | | 0.099 | 0.631 | 22 (lr = 0.01) |
| DICOM | No | 1 | 255 | 0.136 | 0.688 | 21 (lr = 0.01) |
| | No | 3 | 255 | 0.741 | 0.926 | 22 (lr = 0.01) |
| | No | 5 | 255 | 0.767 | 0.943 | 20 (lr = 0.01) |
| | Yes | 1 | 255 | | | |
| | Yes | 3 | 255 | | | |
| | Yes | 5 | 255 | | | |

### Example for Mammography Density

The systems and methods for image reformatting may be applied to preprocess images for a neural network configured to classify breast density in medical images. In some configurations, reformatting may increase the conspicuity for discerning breast density in the images. An overview of an example dataset where some images where used for training, validation, and testing of a neural network's ability to classify breast density is shown in Table 8 and Table 9, where windowed image refers to the reformatted image and full-range DICOM image refers to an original image with 1 input channel used for all cases.

TABLE 8

| | Train | Validation | Test |
|---|---|---|---|
| D1 | 295 | 100 | 100 |
| D2 | 1167 | 400 | 400 |
| D3 | 1184 | 400 | 400 |
| D4 | 293 | 100 | 100 |
| Total | 2939 | 1000 | 1000 |

TABLE 9

| Density | Train | Validation | Test |
|---|---|---|---|
| D1 | 295 | 100 | 100 |
| D2 | 1167 | 400 | 400 |
| D3 | 1184 | 400 | 400 |
| D4 | 293 | 100 | 100 |

The present disclosure has described one or more preferred embodiments, and it should be appreciated that many equivalents, alternatives, variations, and modifications, aside from those expressly stated, are possible and within the scope of the invention.

1. A method for configuring medical imaging data acquired from a patient for processing using a general processing graphic processing unit (GPGPU) architecture, the method comprising:
a) acquiring medical imaging data acquired from a patient using at least one of a magnetic resonance imaging (MRI) system, a computed tomography (CT) system, an ultrasound system, or a positron emission tomography (PET) system and having data characteristics incompatible with processing on the GPGPU architecture, including at least one of bit-resolution, memory capacity requirements for processing, or bandwidth requirements for processing;
b) subjecting the medical imaging data to a system for translating medical imaging data for processing by the GPGPU architecture configured to:
determine a plurality of window level settings using a machine learning network to increase conspicuity of an object in an image generated from the medical imaging data or generate at least two channel image datasets from the medical imaging data;
create translated medical image data using at least one of the window level settings or at least two channel image datasets;
c) processing the translated medical image data using the GPGPU architecture to generate medical images of the patient; and
d) displaying the medical images of the patient.

2. The method of claim 1 wherein determining a plurality of window level settings includes determining a number of channel images using an area under a curve created by an upper bound value of an activation function.

3. The method of claim 2 wherein the activation function includes at least one of a linear function, a tanh function, a sigmoid function, a ReLU function, a leaky ReLU function, or a function that is a combination of these functions.

4. The method of claim 1 wherein the translated medical image data has at least one of at least one of a bit-resolution, a memory capacity requirement for processing, or bandwidth requirement for processing selected to be compatible with the GPGPU architecture.

5. The method of claim 1 further comprising reading a DICOM header data from the medical imaging data and providing the DICOM header data to the machine learning network to determine the plurality of window level settings.

6. The method of claim 1 wherein the window level settings are based upon a quantitative value.

7. The method of claim 6 wherein the quantitative value includes Hounsfield Units.

8. The method of claim 1 further comprising colorizing the channel images by assigning a specific color to each channel image.

9. The method of claim 8 further creating a third channel image to create a RGB reformatted image.

10. A system for translating medical imaging data acquired from a patient for processing using a general processing graphic processing unit (GPGPU) architecture, the system comprising:
a first processor configured to:
acquire medical imaging data acquired from a patient and having data characteristics incompatible with processing on the GPGPU architecture, including at least one of bit-resolution, memory capacity requirements for processing, or bandwidth requirements for processing;
translate medical imaging data for processing by the GPGPU architecture by:
determining a plurality of window level settings using a machine learning network to increase conspicuity of an object in an image generated from the medical imaging data or generate at least two channel image datasets from the medical imaging data;
creating translated medical image data using at least one of the window level settings or at least two channel image datasets;
a second processor having a GPU architecture configured to process the translated medical image data using the GPGPU architecture to generate medical images of the patient; and
a display configured to display the medical images of the patient generated by the GPGPU architecture.

11. The system of claim 10 wherein determining a plurality of window level settings includes determining a number of channel images using an area under a curve created by an upper bound value of an activation function.

12. The system of claim 11 wherein the activation function includes at least one of a linear function, a tanh function, a sigmoid function, a ReLU function, a leaky ReLU function, or a function that is a combination of these functions.

13. The system of claim 10 wherein the translated medical image data has at least one of at least one of a bit-resolution, a memory capacity requirement for processing, or bandwidth requirement for processing selected to be compatible with the GPGPU architecture.

14. The system of claim 10 wherein the first processor is further configured to read a DICOM header data from the medical image data and provide the DICOM header data to the machine learning network to determine the plurality of window level settings.

15. The system of claim 10 wherein the window level settings are based upon a quantitative value.

16. The system of claim 15 wherein the quantitative value includes image intensity values associated with the medical imaging data.

17. The system of claim 10 further comprising colorizing the channel images by assigning a specific color to each channel image.

18. The system of claim 17 further comprising generating a third channel image to create a RGB reformatted image.
